(19) United States
(12) Patent Application Publication
(10) Pub. No.: US 2010/0323907 A1
(43) Pub. Date: Dec. 23, 2010

(54) FROZEN CELL AND TISSUE MICROARRAYS

(76) Inventors: Long Ai Ton-That, Bronx, NY (US); Gavreel Kalantarov, Fort Lee, NJ (US); Ilya Trakht, Bronx, NY (US)

Correspondence Address: DITTHAVONG MORI & STEINER, P.C., 918 Prince Street, Alexandria, VA, 22314, US

(21) Appl. No.: 12/446,575

(22) PCT Filed: Oct. 27, 2006
(86) PCT No.: PCT/US06/60326

§ 371 Date: Apr. 21, 2009

(51) Int. Cl. C40B 30/04; C12N 5/071; G01N 33/53; C12Q 1/68; C40B 50/06; C40B 40/02

(52) U.S. Cl. 506/9; 435/404; 435/395; 435/7.1; 435/7.92; 435/6; 506/26; 506/14

(57) ABSTRACT

The invention is directed to a new composition for making a housing block for cryosectioning comprising agarose and optimal cutting temperature medium. The invention is further directed to new methods for making a frozen section microarray of fresh non-fixed frozen cell or tissue samples that undergo only one freeze-thaw cycle before being used in a biological assay.

### STATEMENT OF GOVERNMENTAL INTEREST

This invention was not made with any Government support.

### BACKGROUND

1. Field of the Invention

The present invention relates to compositions and methods for making frozen cell and tissue microarrays.

2. Description of the Related Art

Tissue microarray technology has recently been developed that allows for the rapid high-throughput profiling of normal and tumor tissue specimens. In addition to allowing the investigator to assess histomorphology, tissue microarrays can be used to analyze the expression of molecules at the DNA, mRNA, and protein levels. Potential applications for tissue microarrays span a broad range and include analysis of the frequency of molecular alterations in large numbers of tumors, exploration of tumor progression, identification of predictive or prognostic factors, and validation of newly discovered genes as diagnostic and therapeutic targets at a speed comparable to DNA microarrays.

A cell or tissue microarray is an ordered array of numerous cell or tissue samples, which is attached onto a single glass slide. Biological tissues useful in the tissue microarray include human tissues, animal tissues and cultured cells or primary cell preparations (e.g. blood cells). The use of microarrays increases the throughput of molecular analyses by simultaneously arraying proteins, nucleic acids, and other biomolecules for treatment and analysis. Cell and tissue microarrays allow for the study of protein expression, nucleic acid hybridization, receptor-ligand interaction, antigen localization and molecular profiling. The slide can be applied for a broad range of in situ assays, including immunohistochemistry, in situ hybridization (FISH-fluorescent in situ hybridization), karyotyping, comparative genomic hybridization (CGH), special stains and in situ PCR.

Recently developed high density tissue microarray technology involves arraying up to 1000 cylindrical tissue cores from individual tumors on a tissue microarray. Kononen J, Bubendorf L, Kallioniemi A, Barlund M, Schraml P, Leighton S, Torhorst J, Mihatsch M J, Sauter G, Kallioniemi O P: Tissue microarrays for high-throughput molecular profiling of tumor specimens. Nat Med 1998, 4:844-847. More than 200 serial sections can then be made from an individual microarray block and used for analysis of DNA, RNA, and/or proteins on a single glass slide. The technology is useful in that it allows rapid analysis of a large number of samples so that the statistical relevance of new markers can be determined in a single experiment. In addition, altered expression levels can be correlated to amplification or deletion events in specific tumor samples using serial sections, thus allowing simultaneous determination of gene copy number and expression analysis of candidate pathogenic genes and suppressor genes. Arrays have been made containing numerous tumor types as well as multiple stages and grades within individual tumor types. This technology has already proven useful for rapidly characterizing the prevalence and prognostic significance of differentially expressed genes identified using cDNA array technology as well as genes involved in cancer development and progression. Tissue microarrays have also been useful in identifying genes that are targets of chromosomal amplification as well as to study the expression patterns of putative tumor suppressor genes. Some technical problems exist with this methodology, however, relating to the fact that the arrayed samples are typically pre-fixed and embedded in paraffin. The quality of the studies performed on sections from tissue array technology may be limited by the fixation methods used on the original sample; stronger fixatives can partially degrade proteins and nucleotides. It is also a problem that previously described methods typically cause specimens to be frozen and thawed more than once which affects cell viability and biological activity.

More recently others have described methods for preparing microarrays of frozen non-fixed cell and tissue samples for cryosectioning. Stephan J P, Schanz S, Wong A, Schow P, Wong W L. Development of a frozen cell array as a high-throughput approach for cell-based analysis, Am J Pathol. 2002 September; 161(3):787-97; Schoenberg Fejzo M, Slamon D J. Frozen tumor tissue microarray technology for analysis of tumor RNA, DNA, and proteins. Am J Pathol. 2001 November; 159(5):1645-50; Salmon et al. U.S. Pat. No. 6,893,837. 2005; and Hoos A, Cordon-Cardo C. Tissue microarray profiling of cancer specimens and cell lines: opportunities and limitations, Lab Invest. 2001 October; 81(10):1331-8, all of which are incorporated herein by reference. All of these methods embedded microarrays of non-fixed frozen tissue or cell samples in Optimal Cutting Temperature compound (hereafter called OCT). OCT exists in liquid form at temperatures above 0° C. This means that OCT housing blocks have to be kept frozen at temperatures below −10° C. However, at these low temperatures blocks of OTC tend to crack and split easily, and the quality of frozen sections prepared from them is not ideal. Moreover, the methods for making microarrays of non-fixed tissues using OCT housing blocks usually means subjecting the tissue to more than one round of freezing and thawing, which affects cellular integrity, viability, histology and reactivity in various biological assays. Cell samples placed into the holes of pre-frozen OCT blocks freeze rapidly which also causes cell damage. Therefore, there is still a need for simple, reliable, and cost-efficient methods for making frozen cell and tissue microarrays that allow good preservation of fresh non-fixed samples for histology, various biologic assays, and high-throughput screening.

### DEFINITIONS

In order to more clearly and concisely describe and point out the subject matter of the claimed invention, the following definitions are provided for specific terms which are used in the following written description and the appended claims.

As defined herein, a “tissue” is an aggregate of cells and non-cellular extracellular components that perform a particular function in an organism and it refers to a combined cellular and non-cellular extracellular material from a particular physiological region. The cells in a particular tissue may comprise several different cell types. A non-limiting example of this would be brain tissue that further comprises neurons and glial cells, as well as capillary endothelial cells and blood cells. The term “tissue” also encompasses a plurality of cells contained in a sublocation on the tissue microarray that may normally exist as independent or non-adherent cells in the organism, for example immune cells, or blood cells. The term “tissue” refers to tissue samples isolated from humans, animals and plants.

As defined herein a “sample of cells” and a “cell sample” refer to a suspension of cells (e.g., from a cell line).

A biological sample as used herein means a cell or tissue sample.

The terms “fixing” and “fixed” are used herein according to their art-accepted meaning and refer to the chemical treatment (including formation of cross-links between proteins and protein denaturation by coagulation) of biological material, which can be accomplished by the wide variety of fixation protocols known in the art (see, e.g., Current Protocols In Molecular Biology, Volume 2, Unit 14, Frederick M. Ausubul et al. eds., 1995). The term “non-fixed” refers to biological samples that have not been chemically modified or treated (e.g. with reagents such as formalin and ethanol).

As defined herein, “a tissue microarray” (hereafter called TMA) is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the array, but merely encompasses a plurality of tissue samples which, in one embodiment, can be viewed using a microscope.

As defined herein, “a cell microarray” (hereafter called CMA) is a microarray that comprises a plurality of sublocations, each sublocation comprising cells, where the morphological features of the cells at each sublocation are visible through microscopic examination.

As defined herein, “microarray of biological samples” includes cell and tissue microarrays.

“Agarose” as defined herein is essentially the neutral gelling fraction of agar, consisting of a linear polymer based on the -(1>3)-β-D-galactopyranose-(1>4)-3,6-anhydro-α-L-galactopyranose units. Agarose is typically high in molecular weight, which is about 120,000 and low in sulphate.

As defined herein, the term “OCT” compound means OCT compound™ (product code 4583) sold by Tissue Tek® containing water-soluble glycols and resins (10.24% polyvinyl alcohol, 4.26% polyethylene glycol and 85.50% non-reactive ingredient). OCT compound is used as a cell or tissue sample matrix for cryostat sectioning at temperatures of −10° C. and below, leaves no residue on slides and eliminates undesirable background during staining procedure. OCT compound exists only in liquid form at temperatures higher than 0° C., and gradually solidifies at temperatures below −20° C. Blocks made of OCT must be handled at −10° C. or below in order to preserve their shape.

As defined herein, the term “Agarose-OCT” means the new composition of the present invention: a composition comprising from about 0.5% to 15% agarose in OCT compound (w/v). Any commercial agarose (i.e., standard agarose or any kind of low melting agarose, including low melting, super low melting and extra low melting agarose) can be used and is available from various companies, including Sigma-Aldrich, Fisher Scientific, or elsewhere. A composition of approximately 3-7% agarose in OCT (w/v) is preferred for making blocks of microarrays of frozen biological samples.

As defined herein, a “housing block” means a block made of OCT or Agarose-OCT compound, or Agar-OCT, in which one or more holes capable of housing a biological sample are made, and which is not yet loaded with biological samples. In the preferred embodiment one or more, usually an array, of holes are made in the housing block with an open end on one surface of the block and the other end of the hole being closed so that the hole is capable of containing a cell or tissue sample. Formats for housing blocks made of Agarose-OCT include All-in-One Master blocks, Unit blocks and Sub-Master blocks.

As defined herein, a “loaded block” is a “housing block”, in which one or more holes are filled with a biological sample.

As defined herein, an “All-in-One Master block” is an individual housing block made of Agarose-OCT (or Agar-OCT) comprising a specific number of holes, which can be any number, preferably from 1 to 72, depending on the size of the holes and the size of the block. All-in-One Master blocks can be in any shape. This kind of block is made in a single mold. FIGS. 2, 4 and 6.

As defined herein, a “Unit block” is a small individual housing block made of Agarose-OCT that typically has fewer than 24 holes, sometimes as few as 1-2 or 4-8 holes. FIG. 3 shows small unit blocks cut from a larger Master block. Unit blocks can be of any shape including, a cube, a column or a bar.

As defined herein, a “Sub-Master block” is a block formed by sticking or gluing together two or more Unit blocks, using as glue any cryosectioning medium compound for cryo-embedding capable of gluing together individual blocks, including but not limited to OCT compound or Agarose-OCT compound.

As defined herein, a “gel-solid” composition/block means the Agarose-OCT composition or a block made from Agarose-OCT that is unfrozen, is in a gel-phase, and is soft and flexible. Agarose-OCT blocks are gel-solid at room temperature and at temperatures of about 0° C.

As defined herein, an “ice-solid” composition/block means the Agarose-OCT composition or a block made from Agarose-OCT that is hard, inflexible and is in frozen form. OCT gradually becomes ice-solid at temperature below about −10° C.

### BRIEF DESCRIPTION OF THE DRAWINGS

The present invention is illustrated by way of example, and not by way of limitation, in the following figures.

FIG. 1: A housing block made of Agarose-OCT compound prior to punching the holes.

FIG. 2: An All-in-One Master housing block made of Agarose-OCT compound after punching holes.

FIG. 3: Individual Unit blocks made of Agarose-OCT with the holes prepared for loading and housing tissue or cell samples.

FIG. 4: A frozen All-in-One Master block loaded with arrays of cell samples.

FIG. 5: A slide containing sections of freshly frozen CMA obtained from a frozen All-in-One Master block and stained with hematoxylin and eosin.

FIG. 6: A frozen All-in-One Master block loaded with arrays of tissue samples.

FIG. 7 A slide containing sections of freshly frozen TMA obtained from a frozen All-in-One Master block and stained with hematoxylin and eosin (H&E).

FIG. 8 Representative microscopic images of sections of fresh non-fixed frozen mouse tissues, which were microarrayed on a housing block made of Agarose-OCT. Frozen sections were stained with H&E. The numbers indicate the magnification of images.

FIG. 9 Representative fluorescent microscopic images of sections of fresh non-fixed frozen mouse tissue microarray, cryosectioned from a housing block made of Agarose-OCT. Frozen sections were stained with mouse monoclonal anti-alpha-smooth muscle actin antibody conjugated with a fluorescent tag. White arrows show locations that are positively-stained with the antibody. The numbers indicate the magnification of images.

FIG. 10 Representative fluorescent microscopic images of sections of fresh non-fixed frozen cell microarray, cryosectioned from a housing block of Agarose-OCT. The cells were from various human cancer cell lines. Frozen sections were stained with human monoclonal antibodies (huMAbs). The binding of huMAbs to the cells was identified by fluorescent-conjugated goat antibodies against light chains (κ or λ) of immunoglobulin. Positive staining is indicated as bright rings or dots surrounding nuclei of the cells, as shown by white arrows. The numbers indicate the magnification of images.

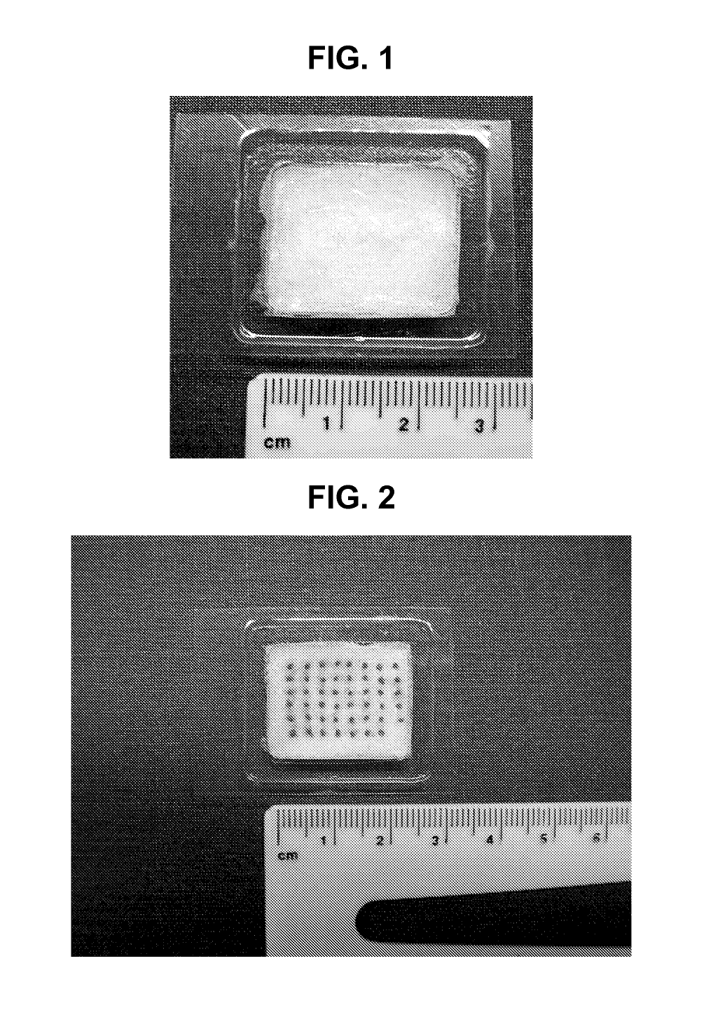

### SUMMARY OF THE INVENTION

Certain aspects of the invention are directed to a new composition comprising on a weight/volume (w/v) basis from about 0.5% to about 15% agarose in OCT. One aspect is directed to the composition comprising from about 3% to 7% agarose in OCT (w/v), which is ideally suited for making housing blocks for cryosectioning cell or tissue microarrays because it is soft and flexible at temperatures from about 0° C. to about 37° C. and it freezes to become hard and inflexible at temperatures below about −10° C. Another aspect is directed to the composition of OCT and agarose, made by (a) mixing from about 0.5% to about 15% agarose in OCT compound w/v, preferably from about 3% to 7% agarose in OCT (w/v), and (b.) heating the mixture of step (a) until a homogeneous material is obtained. Other aspects further comprise making a housing block of OCT agarose by adding the additional steps of (c) pouring the liquid heated Agarose-OCT composition of step (b) into a mold, and (d.) cooling the Agarose-OCT composition until it becomes solid to obtain the housing block. In yet other aspects the housing block further comprises holes disposed therein, preferably in an array, which holes are capable of containing a biological sample.

Certain aspects of the invention are further directed to a new method of preparing a non-fixed, never-frozen cell sample microarray, comprising (a) providing a housing block made of a composition comprising from about 0.5% to 15% agarose in OCT compound (w/v), which housing block further comprises one or more holes disposed in an array that are capable of containing a cell sample, (b) cooling the block until it reaches a temperature of from about +8° C. to about 0° C. and maintaining the block at this temperature until the desired number of holes are filled with cell samples, (c) filling a hole in the housing block with a non-fixed, never-frozen cell sample, (d) repeating step (c) until the desired number of holes are filled with cell samples thereby making a loaded block, (e) gradually cooling the loaded block at a rate of about 1° C. per minute until the block is frozen at a desired temperature, and (f) cryosectioning the loaded block to obtain a non-fixed frozen cell sample microarray.

Another aspect is directed to a new method of preparing a non-fixed, frozen tissue microarray, comprising (a) providing a housing block made of a composition comprising from about 0.5% to about 15% agarose in OCT compound (w/v), which housing block further comprises one or more holes disposed in an array that are capable of containing a frozen tissue sample, (b) cooling the block until it reaches a temperature of at least about −4° C. and maintaining the block at this temperature until the desired number of holes are filled with cell samples, (c) putting liquid OCT compound in a hole in the block, (d) inserting a non-fixed, frozen tissue sample into the hole of step (c) as soon as possible before the liquid OCT compound hardens, (e) repeating steps (c) and (d) until the desired number of holes are filled with non-fixed, frozen tissue samples thereby making a loaded block, (f) freezing the loaded block to a desired temperature, and (g) cryosectioning the loaded block to obtain a non-fixed frozen tissue sample microarray.

Other aspects of the invention include obtaining sections of the cell or tissue microarray for use in a biological assay selected from the group comprising in situ assays, including immunohistochemistry, immunocytochemistry, in situ hybridization, fluorescent in situ hybridization (FISH), karyotyping, comparative genomic hybridization (CGN), special stains and in situ polymerase chain reaction (PCR).

Other aspects of the invention include a composition for making a cell or tissue microarray for cryosectioning, comprising: (a) a housing block made of a compound comprising from about 3% to about 7% agarose in OCT compound (w/v) having an array of holes capable of containing a biological sample disposed therein; and (b) a non-fixed sample of cells or tissue disposed in one or more of the holes in the array of holes in the housing block. An embodiment is also directed to a composition for making a cell or tissue microarray for cryosectioning, wherein the composition is generated by: (a) providing a housing block made of a compound comprising from about 0.5% to about 15% agarose in OCT compound (w/v) having an array of holes capable of containing a biological sample disposed therein; and (b) introducing a non fixed sample of cells or tissue into one or more of the array of holes in the housing block. In some embodiments the cell or tissue sample is frozen; in other embodiments the samples have never been frozen.

### DETAILED DESCRIPTION

In the following description, for the purposes of explanation, numerous specific details are set forth in order to provide a thorough understanding of the present invention. However, a person of skill in the art will know which details can be varied or modified using routine experimentation.

The invention disclosed herein is represented by a number of embodiments. Certain embodiments of the present invention are directed to a new composition made essentially of agarose and OCT compound (hereafter called “Agarose-OCT”), which composition is especially useful for making a housing block for cell or tissue microarrays for frozen sectioning, also called cryosectioning. The new composition is a mixture of agarose and optimal cutting temperature medium (hereafter called “OCT”) with from about 0.5% to 15% agarose in OCT (w/v); preferably from about 3% to 7%.

A typical representative embodiment includes a method of preparing a fresh non-fixed, never-frozen cell sample for microarray analysis. The cell samples are put into the holes made in an array in an unfrozen housing block made of Agarose-OCT comprising preferably from about 3% to 7% agarose in OCT compound (w/v), which block has been cooled to from about +8° C. to about 0° C. The housing block is maintained at this temperature until the desired number of holes are filled with cell samples suspended in a cryoprotective medium thereby making a loaded block. Once the Agarose-OCT block is loaded, it is gradually cooled at a rate of about 1° C. per minute to avoid damaging or destroying the cells until it reaches a desired temperature either for cryosectioning right away or for long term storage. Frozen sections of the loaded block provide a non-fixed frozen cell sample microarray for microarray analysis. Using Agarose-OCT housing blocks and gradual freezing in cryoprotective media optimizes cell bioactivity and histology. This way the cells are frozen only once before being used for a biological assay or cytochemical analysis, thereby preserving the natural condition and viability of the cells. The procedure is discussed in more detail below.

Another embodiment of the invention is directed to a method of preparing a non-fixed, freshly frozen tissue sample for microarray analysis. The frozen tissue samples are loaded into the holes in a frozen Agarose-OCT housing block made preferably of from about 3% to 7% agarose in OCT compound (w/v), which block has been cooled to a temperature of approximately −4° C. or lower to keep the tissue samples frozen. The housing block is maintained at this temperature until the desired number of holes are filled with frozen tissue samples thereby making a loaded block. The loaded block is then frozen, preferably rapidly, to a desired temperature for cryosectioning right away or long term storage. Frozen sections of the loaded block provide a non-fixed frozen tissue sample microarray for microarray analysis. The tissue samples do not thaw during this, thus they are frozen only once before being used for a biological assay. The procedure and variations for making a tissue microarray of fresh never-frozen tissue is discussed in more detail below.

In other embodiments the described methods for making a cell or tissue microarray further include the steps of mounting the frozen cell sample or tissue sample microarray on a glass slide and using it in biological assays including in situ assays, immunohistochemistry, immunocytochemistry, in situ hybridization, fluorescent in situ hybridization (FISH), karyotyping, comparative genomic hybridization (CGH), in situ PCR, and special stains. After the frozen microarray is on the glass slide it can be fixed in acetone cooled to about −20° C. for about 5 to 10 minutes, or in any fixative compatible with the intended biological assay. For example, in U.S. Pat. No. 6,893,837, Slamon et al. demonstrated that a tumor tissue microarray of frozen non-fixed tissues could be used for analysis of RNA using non-radioactive RNA in situ hybridization on array slides that were fixed up to 12 hours or more in 4% paraformaldehyde with excellent preservation of intact RNA. They also showed that a frozen non-fixed tissue array gave excellent results for FISH-based experiments to analyze DNA by fixing cryosections of the tissue microarray in Carnoy's fixative or ethanol. Thus frozen non-fixed tissue and cell microarrays provide excellent target material for the study of DNA, RNA, and proteins by fixing each array slide in a manner specific to the corresponding technique used.

### Housing Blocks of Agarose-OCT for Cryosectioning

The new “Agarose-OCT” composition comprising agarose in an amount of between about 0.5% to about15% in OCT (w/v); preferably from about 3% to about 7%, is made by mixing agarose with liquid OCT and heating the mixture to a temperature of about 50-60° C. until it is homogenized, i.e. the agarose dissolves and mixes thoroughly with the OCT. The new composition is suitable for making housing blocks for cryosectioning as it has characteristics that are compatible with OCT, and it is composed of neutral agents (i.e., agarose and OCT) that do not affect any biological assay.

Agarose-OCT blocks made of 3% to 7% agarose in OCT (w/v) are soft and easily manipulated at wide range of temperatures from 0° C. to about +37° C.; and when frozen the block provides good quality cryosections. Agarose-OCT washes off of a support such as a glass slide in an aqueous solution, or in solvents such as xylene. If agarose is more than about 15% w/v, the Agarose-OCT block is difficult to cryosection. At amounts below about 0.5% w/v agarose in Agarose-OCT, good cryosections can be easily obtained from a frozen block, but an unfrozen block is too soft and wet at room temperature, making it difficult to handle or manipulate the block. Therefore agarose between about 3% and 7% in OCT (w/v) is preferred for making housing blocks for cryosectioning.

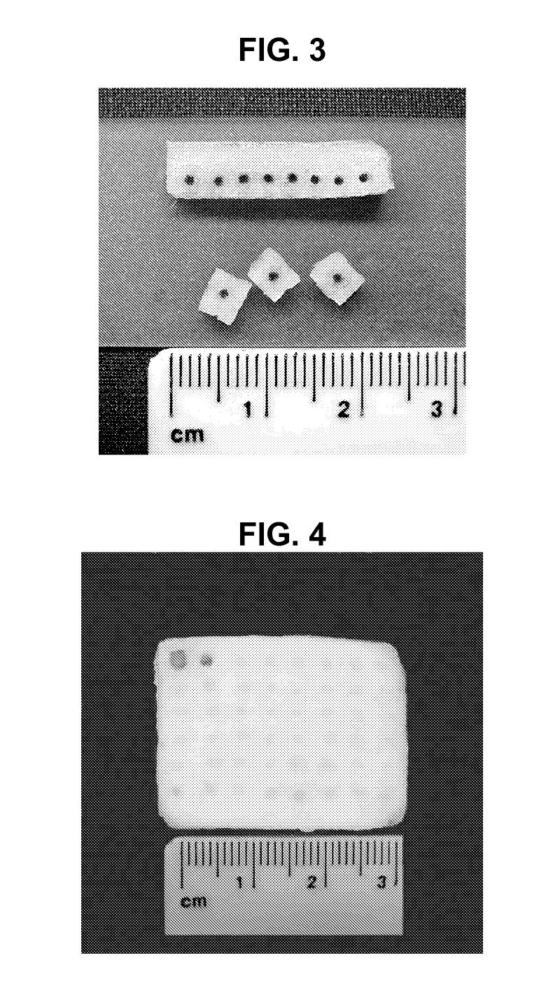

It was unexpected to discover that Agarose-OCT maintains its shape (without melting) at temperatures between about 0° C. and 37° C. At this temperature range it is soft and flexible, which is herein called “gel-solid.” Agarose-OCT is completely ice-solid at temperatures below −10° C. The preferred use of Agarose-OCT is for making housing blocks of any shape for cryosectioning cell and tissue microarrays. In a preferred embodiment a block made of Agarose-OCT has holes capable of containing a biological sample, preferably disposed in an array that makes it easy to determine the site of a particular biological sample for future analysis, for example through a microscope. FIG. 2-FIG. 4.

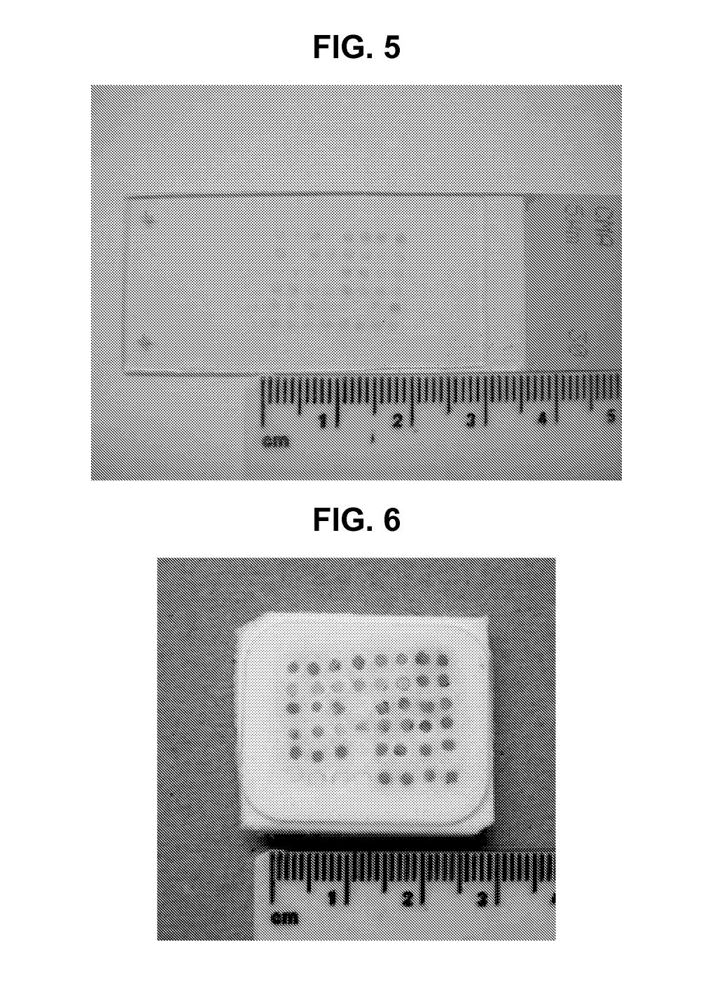

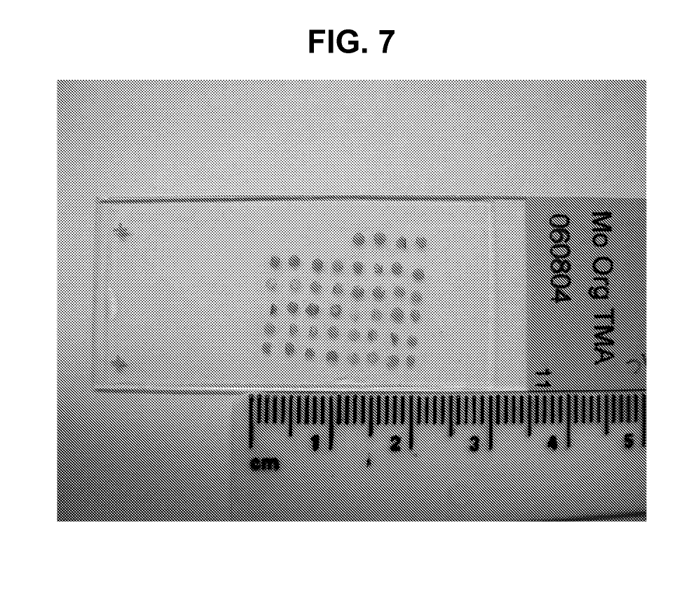

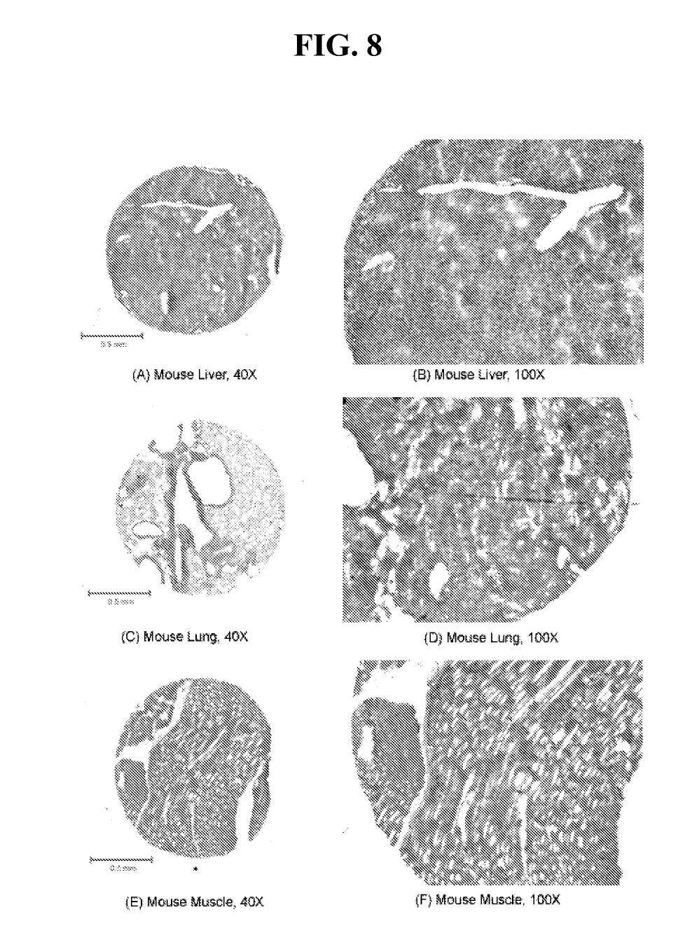

Because Agarose-OCT is firm at room temperature and below, it can be easily handled and manipulated. Examples of different housing blocks and methods for making them are set forth in detail in the Examples. A housing block filled with one or more biological samples is called a “loaded block.” FIG. 5-FIG. 7. Frozen sections of cell or tissue microarrays cut from an Agarose-OCT housing block are prepared according to the methods described below, undergo only one cycle of freezing and thawing before being used in a biological assay. This preserves the freshness and natural biological activity of the sample, which optimizes the accuracy of the assays. FIG. 8-FIG. 10.

Biological samples for cryosectioning can be constructed as arrays (rows and columns), for example arrays of cores of biological samples. To make a microarray the sample is embedded at a specific grid coordinate location in a sectionable housing block. In the past, the process of constructing tissue microarrays typically involved two hollow needle-like punches. One, the “recipient punch”, is slightly bigger and is used to create a hole in a recipient block, typically paraffin or other embedding medium such as OCT compound. The other, the “donor punch”, is smaller and is used to obtain a sample core from a paraffin-embedded donor block or a frozen donor block of tissue. The punches are sized such that the sample core obtained from the donor block (and corresponding to the inner diameter of the donor punch) just fits in the hole created in the recipient block (and corresponding to the external diameter of the recipient punch). Thus the sample snugly fits in the recipient block, and a precise array of sample cores was created.

Previously described methods that used OCT blocks, required that frozen cores of tissue samples be forcibly inserted or “pinned” into the holes of a frozen housing block. Because there is no bonding between the sample core and the housing block, the core has to precisely fit the hole in order to obtain sufficient support for the sample core in the hole of the housing block. This causes some technical problems when making a frozen tissue microarray. Inserting the core into the hole to obtain a tight fit is difficult to accomplish as there is a high risk of breaking either the frozen sample core, the frozen block or both during manipulation. If the core is not snug in the hole of the block, good frozen sections are difficult to obtain. Thus using OCT blocks for cryosectioning is unpredictable and difficult.

By contrast, the new methods for making tissue microarrays eliminate the need for precision-fitting and forcing the tissue sample into a hole in the block. This is because the new methods provide that a hole in the Agarose-OCT block is filled with liquid OCT before the tissue sample is inserted. Thus, when the OCT in the hole freezes and hardens, it provides a unique connection or bond between the tissue sample and the housing block. Similarly, in the new methods for making a cell microarray, a hole in the Agarose-OCT block is filled with cells suspended in cryoprotective medium. When the cryoprotective medium freezes, it likewise bonds with the housing block and supports the cells during sectioning. Cryosections of the cell/tissue microarrays housed in Agarose-OCT blocks are superior and easier to obtain than cryosections of cell/tissue microarrays housed in OCT housing blocks. Another embodiment of the invention is directed to an Agarose-OCT housing block (called a “Sub-Master block”) that is made by gluing together two or more small housing blocks called “Unit blocks” loaded with samples using as glue any compound suitable for cryosectioning including OCT compound™, the medium sold by Instrumedics® Inc. under the name “Cryo-Ge™.” (Cat #ICG-12), and Agarose-OCT or Agar-OCT. Sub-Master blocks make it easy to customize arrays of cell or tissue samples.

It has also been discovered that agar can be substituted for agarose to make a housing block suitable for frozen cell and tissue microarrays. Agar is blended with OCT compound to form an agar-OCT compound, using the same procedure for making the Agarose-OCT compound (i.e. heating to a temperature between about 50° C. and 60° C.). The agar concentration in the Agar-OCT composition is less than about 10% (w/v), preferably 4-5% (w/v). Agarose-OCT blocks are preferred to blocks of Agar-OCT because at the same concentration of agar or agarose, at room temperature a block made of Agar-OCT tends to be wetter and softer than the one made of Agarose-OCT. Also, an ice-solid frozen block of Agar-OCT tends to be harder and more difficult to cryosection. To eliminate these problems, a gel-solid Agar-OCT block needs to be either sufficiently air-dried (which can cause significant shrinkage), or preferably cooled down to temperatures below 0° C., before being manipulated. Further, agar is not as pure as agarose and may contain unidentified impurities that could affect the results of biological assays. Housing blocks for making cryosections of cells and tissues may also be made from a composition including OCT and other gelling agents, such as Phytagel (Sigma, Cat. No. 8169), Agargel (Sigma, Cat. No. A3301), etc. Routine experimentation will determine the ratio of gelling agent to OCT.

### Preparation of A Non-Fixed, Never-Frozen Cell Sample for Microarray Analysis

The new methods for making frozen cell microarrays are superior to other methods for several reasons. They enable fresh, non-fixed cells that were never before frozen to be processed from harvesting through cryosectioning with only one freeze-thaw cycle before being used in a biological assay. According to the new method, an Agarose-OCT housing block is chilled to a temperature range between about +8° C. and 0° C., at which temperature cold cell samples suspended in cryoprotective medium are loaded into holes of the housing block. At these temperatures, the cells loaded into the block do not freeze. Once the Agarose-OCT block is fully loaded with cell samples, it is frozen gradually at a rate of approximately 1° C. per minute to a desired temperature suitable for cryosectioning (about −10° C. or below) or for long-term storage (below −80° C.). This could not have been done with blocks made of entirely of OCT, because OCT blocks should be kept frozen at all times at a temperature below 0° C. Gradual freezing preserves cell viability and causes minimal damage to the antigens and enzymes in the cells.

Loaded blocks made of the preferred formulation of Agarose-OCT (about 3 to 7% agarose in OCT (w/v)) make excellent frozen sections. Routine experimentation will determine the ideal sectioning temperature of Agarose-OCT blocks of varying formulations. The optimal sectioning temperature may vary depending on the ratio of agarose to OCT and the type and size of the cell or tissue sample. However a temperature of at least about −10° C., more preferably between about −15° C. and −20° C. is typically optimal for cryosectioning. This new method results in only one freeze-thaw cycle of cell samples counted from the time when the freshly harvested cell samples are loaded into holes in the housing block until the time when frozen sections are used. FIG. 4 shows a frozen cell microarray block before sectioning; FIG. 5 shows a slide of frozen cell microarray section stained with hematoxylin and eosin.

Previously reported methods for making frozen sections of non-fixed cells or tissue using blocks of OCT require that all steps are done at very low temperatures below −10° C., which causes the small volume of a cell sample to freeze rapidly. Rapid freezing damages the cells and has an adverse effect on the results of subsequent biological assays and on histology. The new methods using Agarose-OCT blocks permit gradual freezing, which keeps the cells intact and permits them to survive both during and after the freezing process. Cell samples that were gradually frozen can even be recovered (thawed) for further rounds of cell culturing. Another improvement is that the methods of the present invention require a smaller quantity of cells, which can be as low as 10 μl of cell suspension per hole, if the hole has a size of about 1 mm in diameter and 5 mm in height.

### An Application of the New Method for Making Fresh Frozen Non-Fixed Cancer Cell Microarrays

We made a cancer cell microarray using fresh, non-fixed cells that had never been frozen according to the methods described above. Frozen sections of the fresh frozen cell microarray (“ffCMA”) embedded in and cut from a housing block of Agarose-OCT were used to screen hybridoma-produced human monoclonal antibodies (huMAbs) for their ability to bind to various cancer antigens using a fluorescent immunocytochemical (ICC) staining method.

Human cancer cell lines for the microarray were purchased from the American Type Culture Collection (ATCC, Manassas, Va., USA). The list of cells is provided in Table 1. All cells were maintained in the respective appropriate culture condition recommended by the ATCC until used for constructing the ffCMA as described herein. Frozen sections of the ffCMA about 5 micrometers in thickness were cut, put onto a glass slide and dried overnight in a refrigerator, before being fixed with cold acetone for 10 minutes the next morning. The slides were stored at −80° C. until used in the ICC assay. Details of the assay are described in Example 3.

TABLE 1

| Cell type | Type of Cancer |
|---|---|
| 1. LnCaP | Prostate |
| 2. PC-3 | Prostate |
| 3. DU-145 | Prostate |
| 4. SK-Br-3 | Breast |
| 5. MCF-7 | Breast |
| 6. ZR-75-1 | Breast |
| 7. MDA-MB-231 | Breast |
| 8. A549 | Lung |
| 9. PaCa-2 | Pancreatic |
| 10. CAPAN-2 | Pancreatic |
| 11. CFPaC-1 | Pancreatic |
| 12. BxPC-3 | Pancreatic |
| 13. SK-Ov-3 | Ovarian |
| 14. OV-CAR | Ovarian |
| 15. SK-Mel | Melanoma |
| 16. SK-Co-1 | Colon |
| 17. CaCo-2 | Colon |
| 18. SW-480 | Colon |
| 19. HT-29 | Colon |
| 20. 5637 | Bladder |
| 21. ScaBer | Bladder |
| 22. Hs143 We | Fibroblast (normal) |
| 23. WS-1 | Fibroblast (normal) |

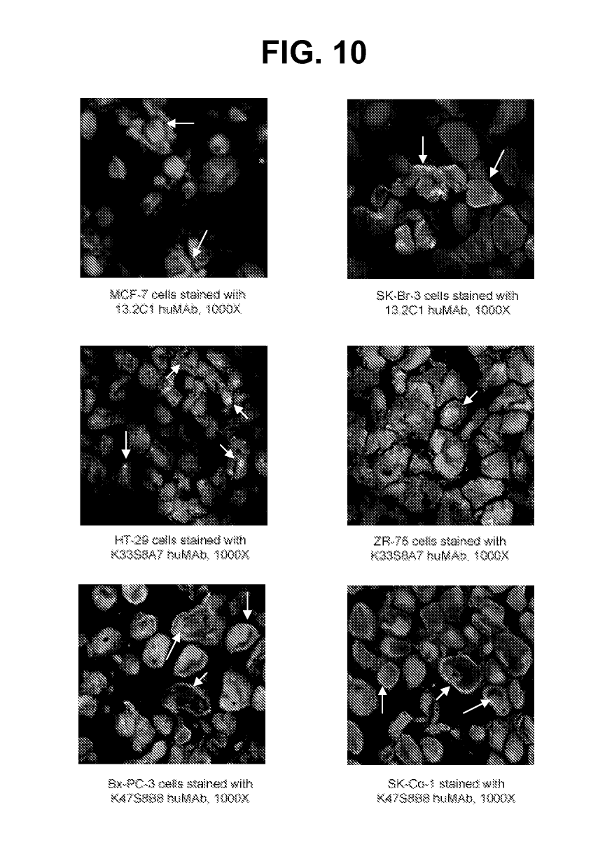

HuMAbs were produced by hybridoma cells, which were generated by fusing human lymphocytes isolated from cancer patients with either MPF-2 cells using methods known in the art. Kalantarov G F, Rudchenko S A, Lobel L, Trakht I. Development of a fusion partner cell line for efficient production of human monoclonal antibodies from peripheral blood lymphocytes. Hum Antibodies (2002); 11(3):85-96) or K6H6/B5 cells (Carroll W L, Lowder J N, Streifer R, Warnke R, Levy S, Levy R. Idiotype variant cell populations in patients with B cell lymphoma (1986). J Exp Med.; 164(5):1566-80), which are incorporated herein by reference. A list of 3 representative huMAbs tested on the cell microarray is provided in Table 2. Target antigens of these hybridoma-produced huMAbs had never before been identified. We tested over 150 different hybridoma-produced huMAbs on sections of ffCMA in the microarray described in Table 1. Representative results and images of three of the huMAbs are shown in Table 2, and in FIG. 10. The results show that the cell microarrays embedded in an Agarose-OCT housing block and processed using the new methods described herein with one freeze-thaw cycle can be used for accurate ICC assays, and that the histological preservation of the cells is very good.

Frozen sections of cell or tissue microarrays cut from loaded Agarose-OCT blocks were typically fixed in acetone pre-cooled to −20° C. for ten minutes or less. Acetone fixation is also used in many conventional methods for making frozen sections of biological samples.

TABLE 2

| | | | Percentage of | | |
|---|---|---|---|---|---|
| | | Tissue | positively stained cells | | |
| | | or | 13.2C1 | K33S8A7 | K47S8B8 |
| | Human cancer | Organ of | huMAb | huMAb | huMAb |
| No. | cell lines | Origin | (μ, κ) | (μ, λ) | (μ, κ) |
| 1 | LnCaP | Prostate | +/− | Negative | Negative |
| 2 | PC-3 | Prostate | >50% | +/− | +/− |
| 3 | DU-145 | Prostate | 20-50% | Negative | <5% |
| 4 | SK-Br-3 | Breast | 10-20% | +/− | 10-20% |
| 5 | MCF -7 | Breast | 10-20% | >50% | 20-50% |
| 6 | ZR-75-1 | Breast | 10-20% | >50% | 20-50% |
| 7 | MDA-MB-231 | Breast | >50% | +/− | Negative |
| 8 | A549 | Lung | 20-50% | Negative | Negative |
| 9 | PaCa-2 | Pancreas | 20-50% | 5-10% | Negative |
| 10 | CAPAN-2 | Pancreas | 20-50% | +/− | Negative |
| 11 | CFPaC-1 | Pancreas | >50% | Negative | Negative |
| 12 | BxPC-3 | Pancreas | >50% | 20-50% | >50% |
| 13 | SK-Ov-3 | Ovary | 20-50% | Negative | Negative |
| 14 | OV-CAR | Ovary | >50% | +/− | Negative |
| 15 | SK-Mel | Melanoma | >50% | +/− | Negative |
| 16 | SK-Co-1 | Colon | >50% | +/− | 20-50% |
| 17 | CaCo-2 | Colon | 5-10% | 5-10% | Negative |
| 18 | SW-480 | Colon | >50% | >50% | Negative |
| 19 | HT-29 | Colon | 10-20% | >50% | Negative |
| 20 | 5637 | Bladder | >50% | Negative | Negative |
| 21 | ScaBer | Bladder | 5-10% | +/− | Negative |
| 22 | Hs143 We | Fibroblast | +/− | +/− | Negative |
| 23 | WS-1 | Fibroblast | +/− | +/− | Negative |

Table 2 shows the results of fluorescent immunocytochemical staining on sections of a freshly frozen cell microarray (ffCMA) comprising human cancer cell lines. All tested antibodies were human monoclonal antibodies (huMAbs) generated by hybridoma technology. Cell samples were incubated with tested huMAbs. The binding of tested huMAbs to the cells was identified by fluorescent-conjugated goat antibodies against light chains (κ or λ) of human immunoglobulins. Each huMAb was tested separately on an individual slide comprising one section of ffCMA.

### Preparation of A Non-Fixed, Pre-Frozen Tissue Sample for Microarray Analysis

Further embodiments of the invention are directed to methods for preparing a tissue microarrays for cryosectioning using samples of fresh non-fixed tissue that have already been frozen (usually snap-frozen right after sampling, for example in the operating room). Frozen sections of the tissue microarrays thus formed are suitable for biological assays. Tissue samples are often already frozen when they come into the laboratory, thus it is important to keep them from thawing out during the formation and cryosectioning of the tissue microarray. In the new method a housing block of Agarose-OCT was chilled to a temperature of at least about −4° C. and maintained at this temperature until it was fully loaded, assuring that the frozen sample inserted in the block stayed frozen. In Example 2C, the block was frozen to −10° C. Before loading the frozen tissue sample in a hole in the block, the hole was filled with liquid OCT compound Immediately thereafter a freshly frozen, non-fixed tissue sample was inserted in the hole before the OCT hardened. The hole was then topped off or covered with a thin layer of liquid OCT. These steps were repeated until the desired number of tissue samples were loaded in the block. The loaded block was then chilled rapidly to a desired temperature either for cryosectioning right away (below −10° C. or lower), or to −80° C. or lower for indefinite storage until future use. Rapid freezing could be done without damaging the tissue because the tissue was already frozen.

To test this method, we made a Tissue Microarray (ffTMA) using freshly frozen (never thawed), non-fixed tissue samples of various mouse organs. Frozen sections were used to determine expression of alpha-smooth muscle actin in the various mouse tissues, using a fluorescent immunohistochemical (IHC) staining method. The tissue microarray included brain, heart, lung, spleen, liver, kidney and testis. Frozen sections of the ffTMA (fresh, frozen tissue microarray) were mounted on glass slides and dried over night in a refrigerator before being fixed with cold acetone for 10 minutes the next morning. The frozen sections were then stored at −80° C. until used in the assay. An FITC-conjugated mouse monoclonal anti-α smooth muscle actin (SMA) antibody purchased from Sigma-Aldrich (Saint Louis, Mo., USA, Cat. No. F3777) was used in the IHC assay, which is described in Example 4.

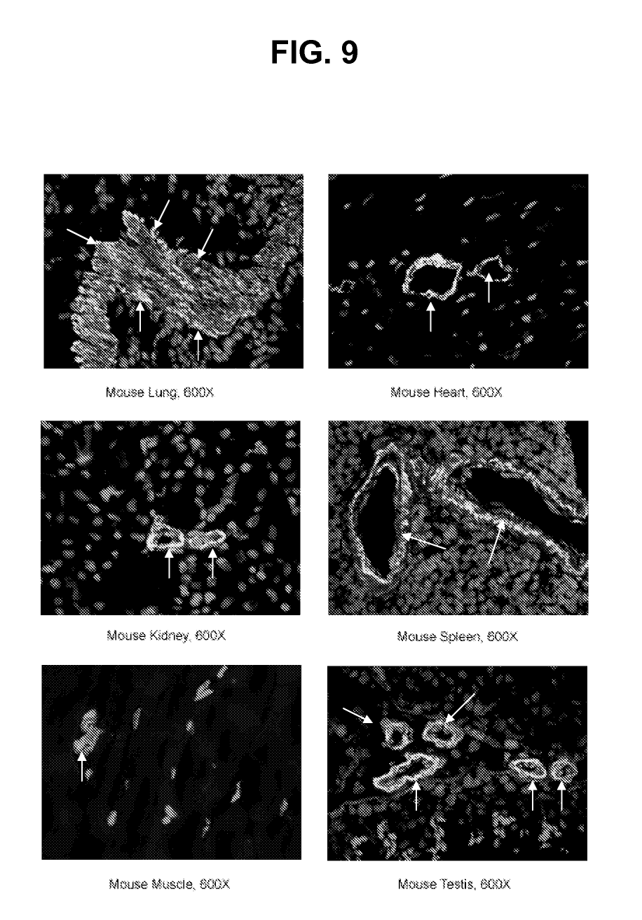

Representative images of the results are shown in FIG. 9. The results showed that the alpha-smooth muscle actin was expressed in tissue samples taken from lung, heart, kidney, spleen, muscle and testis. The tissue processed according to the new methods with acetone fixation had good histological preservation and localized antibody binding.

### Preparation of A Non-Fixed, Never-Frozen Tissue Sample for Microarray Analysis

Further embodiments of the invention are directed to methods for preparing tissue microarrays for cryosectioning using samples of fresh non-fixed tissues and that have never been frozen. If the tissue was obtained using a tissue punch, the tissue can be left in the punch until it is inserted into a hole in an Agarose-OCT housing block that has been chilled to a temperature between about +8° C. and 0° C. At this temperature the tissue is cold but it does not freeze when placed in the hole. A hole in the cold housing block was filled with liquid OCT, and immediately thereafter the fresh non-fixed tissue carried inside the tissue punch was placed in the hole. Liquid OCT was then applied to quickly top off the hole to prevent the tissue from drying out, and the block was immediately snap-frozen to a desired temperature for cryosectioning or for long term storage.

Another modified method is required to make microarrays of fresh, non-fixed, never-frozen tissue slices or pieces that were not obtained using a whole punch. Such non-fixed tissues are soft and would be easily damaged if they were forced into a hole. Therefore to facilitate easy loading of the tissue, a housing block with one hole or a plurality of holes in a straight line is cut in half along the length of the hole(s) making two halves of a block with groves running from top to bottom. Both halves of the block are chilled to a temperature between about +8° C. and 0° C. The fresh, non-fixed tissue was placed in the grove of the cold block and covered with liquid OCT. The second half of the block was then placed over the first half, aligning the groves to reassemble the holes. The reassembled block was immediately snap-frozen to a desired temperature.

### Biological Assays on Non-Fixed Frozen Cell and Tissue Microarrays

Microarrays of freshly frozen non-fixed sections of cell and tissue samples according to the present invention can be used to validate specific biochemical markers for cancer, infectious diseases, and cellular or tissue pathophysiological conditions including hypertrophy, transformation, necrosis, and inflammation. They can also be used for studies, identification and validation of genes at the DNA or RNA levels as well as the expression of individual genes on a protein level that are involved in different human pathologies using nucleic acid probes. Microarrays prepared from freshly frozen non-fixed tissues or cells can be used for in situ PCR, immunohistochemistry or immunocytochemistry, receptor studies and enzymatic studies. The advantage of freshly frozen non-fixed tissue or cell microarrays over formalin fixed microarrays is that freshly frozen tissues and cells preserve the antigens in their natural form thus allowing the detection of specific markers without requiring additional steps to retrieve antigens that can be destroyed by fixation. The new freshly frozen non-fixed cell and tissue microarrays (ffCMA and ffTMA respectively) of this invention also preserve enzyme activity, which can be measured directly on microarray slides. Schellens J P, Vreeling-Sindelarova H, Frederiks W M. Electron microscopic enzyme histochemistry on unfixed tissues and cells. Bridging the gap between LM and EM enzyme histochemistry. Acta Histochem. 2003; 105(1):1-19; Kugler P. Enzyme histochemical methods applied in the brain. Eur J Morphol. 1990; 28(2-4):109-20, incorporated herein by reference. Freshly frozen CMA and TMA also preserve receptor binding activity of membrane and intracellular receptors.

The fact that the cell and tissue microarrays prepared according to the new methods only involve one freeze thaw cycle before a biological assay is done, means that there is minimal damage to cellular architecture, antigens, antigenic epitopes, nucleic acid structure, and enzyme and receptor activity. By contrast, the previously known techniques described in the literature based on using blocks of commercially available OCT compound, may involve more than one round of freezing/thawing of cell or tissue samples.

The cell and tissue samples used in the new methods described here are not fixed until after cryosectioning. This means that they can be fixed in any way that the desired protocol for the assay suggests to optimize/preserve biological activity of the molecule being assayed. In the examples below, the frozen sections were lightly fixed in cold acetone for not more than 10 minutes before being processed for immunocytochemistry or immunohistochemistry. With the present methods, different molecules of interest from a single tissue microarray can be evaluated in mirror or adjacent sections on the same slide or on different slides under optimal conditions (e.g., a first section fixed for the evaluation of polynucleotides and a second section from the same microarray fixed for the evaluation of polypeptides). Another benefit of fixing sections after the samples are arrayed on a slide, is the uniform fixation across the array panel thereby decreasing signal variability that is associated with inconsistent fixation.

The mechanism of action of acetone is unknown, although acetone is classified as a coagulant fixative with methanol and ethanol. Acetone (pre-cooled to −20° C.) has been used successfully as a fixative for frozen tissue and as a dehydrating agent in tissue processing by many researchers. While acetone may brittleness in tissue if exposure is prolonged, the short exposure time of 10 minutes did not cause this problem. It was shown that RNA can be successfully extracted from tissues and cells treated with acetone or methanol. Goldsworthy S M, Stockton P S, Trempus C S, Foley J F, Maronpot R R. Effects of fixation on RNA extraction and amplification from laser capture microdissected tissue. Mol Carcinog. 1999 June; 25(2):86-91; Benchekroun M, DeGraw J, Gao J, Sun L, von Boguslawsky K, Leminen A, Andersson L C, Heiskala M. Impact of fixative on recovery of mRNA from paraffin-embedded tissue. Diagn Mol Pathol. 2004 June; 13(2):116-25, incorporated herein by reference. Acetone has been employed as a fixative in the acetone-methylbenzoate-xylene (AMEX) technique, which showed better histological preservation than is possible to obtain in frozen sections that are completely non-fixed. Sato Y, Mukai K, Watanabe S, Goto M, Shimosato Y. AMEX method. A simplified technique of tissue processing and paraffin embedding with improved preservation of antigens for immunostaining. Am J Pathol 1986; 125: 431-435, incorporated herein by reference. It has also been shown that labile lymphocyte membrane antigens in the acetone-fixed samples retain reactivity. Fixation methods used or adapted from traditional paraffin arrays can be used with the present methods if desired by a person of ordinary skill in the art. (See, e.g. U.S. Pat. Nos. 6,103,518, 6,258,541 and 6,251,601, which are incorporated herein by reference).

### Examples

### Example 1

Protocol for Making a Block of Fresh Frozen Cell Microarrays (ffCMA)

### A. Supplies:

Tissue-Tek® O.C.T. Compound (Fisher Scientific, Cat. No. NC9418069)

Agarose (Fisher Scientific, Cat. No. BP 1356-500)

Disposable vinyl specimen molds: Tissue-Tek® Cryomold® Standard (25 mm×20 mm×5 mm) (Fisher Scientific, Cat. No. NC9643511)

18G needles or punchers

B-D® 1 cc U-100 Insulin Syringe (Becton Dickinson, Cat. No. 329410)

Cells freshly harvested from the cell culture

### B. Preparing the Housing Block as an All-in-One Master Block:

1. We prepared Agarose-OCT compound: by blending 5% agarose with OCT (w/v)), and heating the mixture up to 50° C.-60° C. until a homogeneous material is obtained.

2. Place the above mixture in a vinyl specimen mold of any desired shape and size to cast blocks of Agarose-OCT. After cooling to about room temperature the housing block prepared from Agarose-OCT compound becomes elastic and soft, but still maintains its shape so that holes can be made in the blocks.

### Notes:

“Housing block” means any block that is not loaded with tissue/cell samples. In a preferred embodiment, the housing block has one or more holes disposed in it capable of housing a biological sample. In another preferred embodiment, the holes are arranged in an array. Once one or more holes in the housing block are filled with a biological sample, the block is called a “loaded block.”
Housing blocks can be made in different sizes and shapes depending on the format requirement. After casting, a housing block can be cut into smaller blocks at room temperature for making Unit blocks as is described below.

3. Holes can be made in the gel-solid block, using any method known in the art, including by punching holes using a puncher such as that used for making holes in paraffin-based tissue microarrays, or a needle with a desired size.

### Notes:

The number of holes in the housing block can vary, depending on desirable formats, the size of holes and the size of block (i.e. the size of mold). There may be 12, 24, 36, 48, 72, 96 or any other customized number of holes per block.
The size of the hole for both tissue and cell microarrays (TMA and CMA) may be any size, preferably between about 0.5 mm and 3 mm in diameter for tissue samples and less than about 1 mm for cell samples.

4. Once holes are made in the housing block it is ready for loading the cell or tissue samples, although it has to be cooled to the appropriate temperature as described in the methods for making cell and tissue microarrays. Alternatively it can be wrapped with vinyl film to prevent drying and stored at temperature between 0° C. and +8° C. for future use.

C. Preparation and Loading of a Cell Sample onto the Housing Block:

We processed cells from the cell lines listed in Table 1 according to the following basic method:

1. Cells were harvested, for example, from T-flasks or tissue culture dishes using EDTA/PBS solution, Trypsin/PBS solution or a combination of both.

2. Cells were then washed and resuspended in any cold solution that is specifically designed for cryopreservation of the cells, and kept on ice. The cells stay cold but do not freeze on ice. Suitable freezing solutions include solutions containing 10% DMSO in Fetal Calf or Bovine Serum (FCS); or 10% DMSO, 20% Fetal Calf or Bovine Serum and 70% DMEM, RPMI-1640 or any other culture media.

3. An Agarose-OCT housing block with holes in an array was placed on ice and chilled to 4° C. and cell samples were loaded into holes in the block using an insulin syringe. Notes:

### In a Preferred Embodiment, Each Specimen is Made in Duplicate or Triplicate.

4. The loaded block was then gradually frozen at rate of 1° C. per minute to a desired temperature in a controlled freezer, or in a Styrofoam box or other insulated box which is then placed into a freezer (−80° C.) until cryosectioning.

### Note:

This freezing method was successfully used for cryopreservation of cell cultures. Viability of the cells loaded in the block was preserved using this method.

5. The loaded block was ready for cryosectioning after about 12 hrs. The block can be preserved at −80° C. indefinitely until future use.

6. After cryosectioning, the frozen microarray sections were mounted onto a glass slide. We dried the slides overnight in a +4° C. refrigerator. Routine experimentation and the protocol required for the biological assay will determine whether variations should be made in processing the frozen sections. The next morning, our slides were fixed in cold acetone (−20° C.) for no more than 10 min before being used for immunocytochemistry assays or they were stored at −80° C. until future use. Other fixatives can be used based on the biological assay to be done.

### Note:

Good cryosections as thin as 3 μm can be obtained from the frozen blocks made of Agarose-OCT compound; the cryosections used for biological assays are usually about 5 μm thick.
   D. Block Formats and Alternative Methods for Preparation of ffCMA Blocks

Housing blocks made of Agarose-OCT may have different formats:
a. “All-in-One Master housing blocks” are big individual blocks having a specific number of holes, which can be any number including 12, 24, 36, 48, 72, 96 or more.
b. “Unit housing blocks” are small individual blocks, which typically have between 1 and 12 holes.
c. “Sub-Master housing blocks” are constructed from individual “Unit housing blocks”, which are assembled in an array and bonded together with OCT compound or Agarose-OCT or any other compound for cryo-embedding.
   Methods of Making Sub-Master Blocks of ffCMA from Unit Housing Blocks:

Unit housing blocks are prepared in molds of the desired shape (for example cubic/bar/columned-shaped molds), using the same protocol as described for All-in-One Master blocks. Alternatively, smaller unit blocks can be cut from a larger all in one block. A unit block that is loaded with one or more cell samples (called a loaded Unit block) is made using the same protocol as described for All-in-One Master blocks. To make the Sub-Master block, two or more frozen loaded Unit blocks were arrayed and glued together for example by regular OCT or Agarose-OCT. The assembly of frozen loaded Unit blocks was typically done in a −10° C. cold chamber (e.g., a Styrofoam box containing the cold vapors from dry ice or liquid nitrogen). The Sub-Master block once assembled was then immersed into liquid nitrogen and stored at −80° C. until cryosectioning. This customized sub-master block can be designed to any specification or need.

### Example 2

Protocol for Making a Block of Freshly Frozen Tissue Microarrays (ffTMA)

### A. Supplies:

Tissue-Tek® O.C.T. Compound (Fisher Scientific, Cat. No. NC9418069)

Agarose (Fisher Scientific, Cat. No. BP 1356-500)

Disposable vinyl specimen molds: Tissue-Tek® Cryomold® Standard (25 mm×20 mm×5 mm) (Fisher Scientific, Cat. No. NC9643511)

Punchers: Premier Uni-Punch Disposable Biopsy Punch (Delasco), with diameter sizes of 1.5 mm (Cat. No. UNI/25S/15) and 2.5 mm (Cat. No. UNI/25S/25)

1-cc syringe with a 20G needle

Fresh or snap-frozen tissues

### B. Preparing the All-in-One Master Block:

1. Prepare a master housing block made of Agarose-OCT compound as described in Example 1.

### Note:

The number and size of holes on the housing block may vary, depending on format. The housing block can have any number of holes.
The size of holes is typically between 1 mm and 3 mm in diameter.
2. The housing block is ready for loading the tissue samples. Alternatively, it can be wrapped for example with vinyl film and stored at +4° C. until future use.
   C. Preparation and Loading of Fresh, Snap-Frozen Tissues onto the Housing Block:
1. Non-fixed tissues freshly sampled using any method known in the art and never frozen were cut in small strips of a desired size, preferably about 1 mm in width and about 3-5 mm in length. Strips of tissue specimens were placed into labeled cryo-tubes and snap-frozen in liquid nitrogen. Once snap-frozen they are ready to be loaded into a housing block or stored at −80° C. until future use.

### Note:

Very often tissue specimens supplied to the laboratory pre-frozen, because snap-freezing specimens immediately after removing them from the body is a common and accepted practice.
If pre-frozen tissue samples are used, the samples are typically punched to obtain a frozen core, for example using a Uni-Punch Disposable Biopsy Punch. The tissue-cores can be left frozen inside the punch, which is then used as a carrier to load the frozen cores into holes disposed in the housing block.
2. Right before loading frozen tissue samples onto a housing block, the housing block was placed in a cold chamber chilled to −10° C. (for example, a Styrofoam box containing vapors of dry ice or liquid nitrogen). The housing block was kept in the cold chamber during the loading procedure. The snap-frozen tissue samples were placed on dry ice or in liquid nitrogen to keep them frozen until they were loaded into the holes.
3. A hole in the housing block was filled with regular liquid OCT, using a 1-cc syringe (for example with a 20G needle); then the frozen-tissue strip or core was immediately inserted into the filled hole before the OCT hardened. This step was repeated until the desired number of holes were filled.
4. The tissue-loaded block (hereafter called as loaded block) was then covered with regular liquid OCT, and either put it into liquid nitrogen or placed directly into a freezer (−80° C.) for at least 1 hour.
5. The loaded block is ready for cryo-sectioning after one hour at −80° C. or it can be stored at −80° C. until future use.
6. After cryosectioning, the frozen sections were mounted onto a glass slide and dried overnight in a +4° C. refrigerator. The next morning, the slides were fixed in cold acetone (−20° C.) for no more than 10 min and stored at −80° C. until future use.

### Note:

Cryo-sections as thin as 3 nm can be obtained from the frozen blocks made of Agarose-OCT compound; the cryosections used in research are usually 5 nm thick.
   D. Procedure for Making Sub-Master Blocks from Multiple Unit Blocks Containing Fresh Pre-Frozen Tissue Samples:

### Method 1: This Method is Typically Used to Assemble a Large Block (Sub-Master Block) From Several Frozen Unit Blocks (in Ice-Solid Form) Containing Fresh Non-Fixed Frozen Tissue Specimens to Make an Array.

Each individual Unit housing block was prepared and loaded with the tissue as described above. Blocks were then frozen in liquid nitrogen and stored at −80° C. until use. Where it is desired to make an array of specimens that are stored in two or more small blocks, a Sub-Master block is made as follows:

At the time of assembly, all frozen small tissue sample-loaded Unit blocks were arrayed and glued together by regular OCT compound, however Agarose-OCT and any other compound for cryo-embedding can be used. The assembly of frozen loaded Unit blocks was done entirely in a cold chamber (such as a Styrofoam box containing the vapors of dry ice or liquid nitrogen) at a temperature that keeps the sample frozen, for example −10° C. The Sub-Master block once assembled was immersed in liquid nitrogen and stored at −80° C. until cryostat sectioning.

### Method 2: This Method is Used to Embed Fresh Non-Fixed, Never-Frozen Tissue Samples

Fresh, non-fixed, never-frozen tissue cannot be inserted into a hole filled with OCT compound the way frozen (hard) tissue samples were inserted. Therefore a modified Unit housing block was used. The housing block was evenly cut along the central axis of the hole(s) to obtain 2 halves at room temperature, each of which has a U-shaped groove(s) made by cutting through the block along the length of the hole. The fresh non-fixed, never-frozen tissue specimen was placed in the groove(s) of hole(s) of one half of the Unit housing block and covered with regular liquid OCT compound. Then the other half of the cut block was placed over the first half, thereby re-assembling the hole. The loaded block was then snap-frozen at −80° C. or in liquid nitrogen. The frozen loaded Unit blocks were stored at −80° C. until use.

### Method 3: This Method is Used When Housing Blocks Are in Ice-Solid Form and Tissue Specimens Supplied Are Already Frozen in Advance (Pre-Frozen)

An ice-solid housing block (either All-in-One or Unit block) is prepared as described above in the Method 1. The pre-frozen tissues are punched to obtain frozen tissue cores, for example using a 1.5-mm Uni-Punch Disposable Biopsy Punch. The frozen cores are then loaded into holes of ice-solid housing block to make a frozen tissue-loaded block, using the procedure described above. The frozen loaded blocks are then frozen in liquid nitrogen and stored at −80° C. until use. Where it is desired to make an array of specimens that are stored in two or more small Unit blocks, a Sub-Master block is made using the procedure described above in Method 1.

### Example 3

Immunohistochemistry of Frozen Sections of Freshly Frozen Cancer Cell Microarrays (ffCMA)

Frozen sections of freshly frozen cancer cell microarrays (ffCMA) embedded in an Agarose-OCT housing block were obtained using the methods of the present invention used for immunocytochemistry to screen human monoclonal antibodies. A frozen section microarray of ffCMA of human cancer cell lines embedded in and cut from a housing block of Agarose-OCT was used to screen hybridoma-produced human monoclonal antibodies (huMAbs) for their ability to bind to various cancer antigens expressed by the various cancer cells in the microarray using a fluorescent immunocytochemical (ICC) staining method.

The procedure for the immunocytochemical assay to test the binding of huMAbs to ffCMA sections included:

(a) 1 hour blocking with a solution of 5% Bovine Serum Albumin in PBS.

(b) 1 hour incubation with tested huMAbs,

(c) washing in PBS,

(d) 30 minute incubation with commercial FITC-conjugated goat anti-human either lamda (λ) (Caltag, Burlingame, Calif., USA, Cat. No. H16501) or kappa (K) (Caltag, Burlingame, Calif., USA, Cat. No. H16001),

(e) washing in PBS,

(f) counter-staining with Propidium Iodide (BD Pharmingen, San Jose, Calif., USA, Cat. No. 550825)

(g) washing with PBS,

(h) coating the slide of the stained section with mounting media (Fisher Scientifics, USA, Cat. No. BMM−01) and

(i) covering the section with a glass cover for microscopic analysis.

### Example 4

Immunohistochemistry of Frozen Sections of Fresh, Non-Fixed, Pre-Frozen Tissue Microarrays (ffCMA)

Frozen sections of fresh, non-fixed pre-frozen tissue microarrays (ffTMA) embedded in an Agarose-OCT housing block made according to the methods of the present invention were used for immunocytochemistry. Frozen sections of ffTMA were used to assay expression of alpha-smooth muscle actin in mouse tissues, using a fluorescent immunohistochemical (IHC) staining method. An Agarose-OCT block of ffTMA was constructed, using tissues samples from various mouse organs, including brain, heart, lung, spleen, liver, kidney and testis.

Sections of ffTMA obtained by cryo-sectioning a block of ffTMA, were mounted on a glass slide and dried over night in a refrigerator before being fixed with cold acetone for 10 minutes the next morning. The frozen sections were then stored at −80° C. until used in the assay. An FITC-conjugated mouse monoclonal anti-alpha (a) smooth muscle actin (aSMA) antibody purchased from Sigma-Aldrich (Saint Louis, Mo., USA, Cat. No. F3777) was used.

The procedure for the immunocytochemical assay for staining these ffTMA sections with the FITC-conjugated anti-aSMA antibody briefly includes,

(a) 1 hour blocking with Fab Fragment Goat Anti-Mouse IgG (Jackson Immunoresearch Laboratories, West Grove, Pa., USA, Cat. No. 115-007-003). It is important to note that the tissue microarray was kept frozen until this step.

(b) washing in PBS,

(c) 1 hour blocking with a solution of 10% Fetal Bovine Serum in PBS,

(d) 30 minutes incubation with FITC-conjugated anti-aSMA antibody,

(e) washing in PBS,

(f) counter-staining with Propidium Iodide (BD Pharmingen, San Jose, Calif., USA, Cat. No. 550825),

(g) washing with PBS,

(h) coating the slide of stained section with mounting media (Fisher Scientifics, USA, Cat. No. BMM−01), and

(j) covering the section with a glass cover for microscopic analysis.

### Example 5

### Background Information on Agar and Agarose

Agarose is essentially the neutral gelling fraction of agar, consisting of a linear polymer based on the -(→3)-β-D-galactopyranose-(1→4)-3,6-anhydro-α-L-galactopyranose units. Agarose is typically high in molecular weight, which is about 120,000 and low in sulphate.

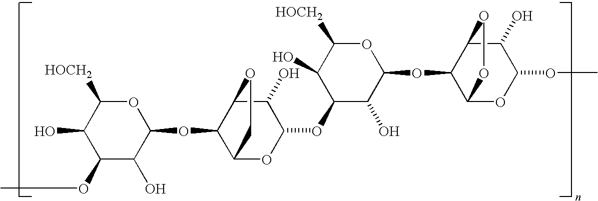

### Structure of Agarose

Agarose is, in practice, purified from agar or agar-bearing marine algae. Agarose forms a gel matrix that is nearly ideal for diffusion and electrokinetic movement of biopolymers. It is routinely used for analysis of nucleic acids by gel electrophoresis or blotting (Northern or Southern) such as gel electrophoresis for separating DNA (Borst P. Ethidium DNA agarose gel electrophoresis: how it started. IUBMB Life. 2005 November; 57(11):745-747), hybridization methods (Lanciotti R S. Molecular amplification assays for the detection of flaviviruses. Adv Virus Res. 2003; 61:67-99; Kroczek R A. Southern and northern analysis. J Chromatogr. 1993 Aug. 25; 618(1-2):133-145). It is also applied for protein analysis such as immunoelectrophoretic methods (Gochman N, Burke M D. Electrophoretic techniques in today's clinical laboratory. Clin Lab Med. 1986 September; 6(3):403-426; Kyle R A. Sequence of testing for monoclonal gammopathies. Arch Pathol Lab Med. 1999 February; 123(2):114-118) and immunodiffusion methods (Smalley D L, Mayer R P, Bugg M F. Capillary zone electrophoresis compared with agarose gel and immunofixation electrophoresis. Am J Clin Pathol. 2000 September; 114(3):487-488; Litwin C M, Anderson S K, Philipps G, Martins T B, Jaskowski T D, Hill H R. Comparison of capillary zone and immunosubtraction with agarose gel and immunofixation electrophoresis for detecting and identifying monoclonal gammopathies. Am J Clin Pathol. 1999 September; 112(3):411-7).

Agar is a polysaccharide complex obtained through bleaching and hot water extraction of agarocytes from the red alga Rhodophyceae, found in the Pacific and Indian Oceans and in the Sea of Japan. The genera Gelidium, Acanthopeltis, Ceramium, Pterocladia and Gracilaria predominate in agar production. Agar is composed of about 70% agarose and 30% agaropectin (Scott T and Eagleson M. Concise Encyclopedia: Biochemistry, 2nd Ed., Walter de Gruyter, New York, 1988, p. 18; Budavari S., Ed. Merck Index, 12th Ed., Merck & CO., INC., New Jersey, 1996, No. 182, p. 34).

Agar has a major use in microbiology and bacteriology to make solid culture media for microorganisms. Agar is also used in other biological methods such as hybridization methods and it does not interfere with these biological assays. (Bolton E. T., McCathy B. J. A general method for the isolation of RNA complementary to DNA. Proc Natl Acad Sci USA. 1962 August; 48:1390-1397; Humm D G, Humm J H. Hybridization of mitochondrial RNA with mitochondrial and nuclear DNA in agar. Proc Natl Acad Sci USA. 1966 January; 55(1):114-119; Hansen J N, Pheiffer B H, Hough C J. Hybrid isolation by recovery of RNA-DNA hybrids from agar using S1 nuclease. Nucleic Acids Res. 1974 June; 1(6):787-801), gel electrophoresis methods (Viljoen C D, Wingfield B D and Wingfield M J. Agar, an alternative to agarose in analytical gel electrophoresis. Biotechnology Techniques. 1993 October, 7(10): 723-726), or in some techniques of embedding tissue samples for histological methods (Lund H Z, Preliminary embedding in agar-agar. Histo-logic 1972; 2: 21; Cook R W, Hotchkiss G R. A method for handling small tissue fragments in histopathology. Med Lab Sci 1977; 34: 93-94; Wigglesworth V B. A simple method for cutting sections in the 0.5 to 1 m range, and for sections of chitin. Quart J Micr Sci 1959; 100: 315-320; Engen P. Double embedding again. Stain Techno., 1974; 49: 375-380)

What is claimed is:

1. A composition comprising on a weight/volume (w/v) basis from about 0.5% to about 15% agarose in OCT.

2. The composition of claim 1, comprising from 3% to 7% agarose in OCT (w/v).

3. The composition of claim 1, wherein the composition is soft and flexible at temperatures from about 0° C. to about 37° C.

4. The composition of claim 1, wherein the composition is hard and inflexible at temperatures below about −10° C.

5. The composition of claim 1, wherein the composition is suitable for cryosectioning.

6. The composition of claim 1, formed into any shape and any size.

7. The composition as in claim 1, formed into a housing block for cryosectioning a biological sample.

8. A housing block for cryosectioning a biological sample, made of a composition comprising from about 0.5% to 15% agarose in OCT (w/v), which housing block comprises one or more holes disposed therein capable of containing a biological sample.

9. The housing block of claim 8, comprising from about 3% to 7% agarose in OCT (w/v).

10. The block of claim 8, further comprising a biological sample disposed in one or more of the holes in the block.

11. The housing block of claim 10, wherein the biological sample is a non-fixed, never-frozen cell sample, a non-fixed never-frozen tissue sample, or a non-fixed frozen tissue sample.

12. Frozen sections cut from the housing block of claim 10 used in a biological assay selected from the group comprising in situ assays, including immunohistochemistry, immunocytochemistry, in situ hybridization, fluorescent in situ hybridization (FISH), karyotyping, comparative genomic hybridization (CGN), special stains and in situ polymerase chain reaction (PCR).

13. The housing block of claim 8, wherein the holes are arranged in an array.

14. A composition comprising OCT and agarose, made by
a. mixing from about 0.5% to about 15% agarose in OCT compound w/v, preferably from about 3% to 7% agarose in OCT (w/v), and
b. heating the mixture of step (a) until a homogeneous material is obtained.

15. A housing block for cryosectioning comprising about from 0.5% to 15% agarose in OCT compound (w/v), made by
a. mixing from about from 0.5% to 15% agarose in OCT compound (w/v),
b. heating the mixture of step (a) a homogeneous material is obtained,
c. pouring the Agarose-OCT composition of step (b) into a mold, and
d. cooling the Agarose-OCT composition until it becomes solid to obtain the housing block.

16. The housing block of claim 15, wherein the amount of agarose is from about 3% to 7% agarose in OCT (w/v).

17. The housing block of claim 15, made by a process further comprising the step of making one or more holes in the block of step (d), preferably in an array, which holes are capable of containing a biological sample.

18. A method of preparing a non-fixed, never-frozen cell sample microarray, comprising
(a) providing a housing block made of a composition comprising from about 0.5% to 15% agarose in OCT compound (w/v), which housing block further comprises one or more holes disposed in an array that are capable of containing a cell sample,
(b) cooling the block until it reaches a temperature of from about +8° C. to about 0° C. and maintaining the block at this temperature until the desired number of holes are filled with cell samples,
(c) filling a hole in the housing block with a non-fixed, never-frozen cell sample,
(d) repeating step (c) until the desired number of holes are filled with cell samples thereby making a loaded block,
(e) gradually cooling the loaded block at a rate of about 1° C. per minute until the block is frozen at a desired temperature, and
(f) cryosectioning the loaded block to obtain a non-fixed frozen cell sample microarray.

19. A method of preparing a non-fixed, frozen tissue microarray, comprising
(a) providing a housing block made of a composition comprising from about 0.5% to about 15% agarose in OCT compound (w/v), which housing block further comprises one or more holes disposed in an array that are capable of containing a frozen tissue sample,
(b) cooling the block until it reaches a temperature of at least about −4° C. and maintaining the block at this temperature until the desired number of holes are filled with cell samples,
(c) putting liquid OCT compound in a hole in the block,
(d) inserting a non-fixed, frozen tissue sample into the hole of step (c) as soon as possible before the liquid OCT compound hardens,
(e) repeating steps (c) and (d) until the desired number of holes are filled with non-fixed, frozen tissue samples thereby making a loaded block,
(f) freezing the loaded block to a desired temperature, and
(g) cryosectioning the loaded block to obtain a non-fixed frozen tissue sample microarray.

20. The method of claim 18 or claim 19, wherein the desired temperature is either about −10° C. to −20° C. for cryosectioning, or about −80° C. for storing the loaded block for an indefinite period until cryosectioning.

21. The method of claim 18 or claim 19, further comprising the steps of
a) mounting the frozen cell sample or tissue sample microarray on a glass slide.

22. The method of claim 21, further comprising the steps of
a) fixing the frozen cell sample microarray or tissue sample microarray on the glass slide in acetone cooled to about −20° C. for about 5 to 10 minutes.

23. The method of claim 21, wherein the cell sample or tissue sample microarray is used in a biological assay selected from the group comprising in situ assays, including immunohistochemistry, immunocytochemistry, in situ hybridization, fluorescent in situ hybridization (FISH), karyotyping, comparative genomic hybridization (CGN), special stains and in situ polymerase chain reaction (PCR).

24. The method of claim 18 or claim 19, wherein the composition comprises from about 3% to 7% agarose in OCT (w/v).

25. A composition for making a cell or tissue microarray for cryosectioning, comprising:
(a) a housing block made of a compound comprising from about 3% to about 7% agarose in OCT compound (w/v) having an array of holes capable of containing a biological sample disposed therein; and (b) a non-fixed sample of cells or tissue disposed in one or more of the holes in the array of holes in the housing block.

26. A composition for making a cell or tissue microarray for cryosectioning, wherein the composition is generated by: (a) providing a housing block made of a compound comprising from about 0.5% to about 15% agarose in OCT compound (w/v) having an array of holes capable of containing a biological sample disposed therein; and (b) introducing a non fixed sample of cells or tissue into one or more of the array of holes in the housing block.

27. The composition as in claim 26, wherein the non-fixed cell or tissue sample is not frozen when it is put into a hole in the housing block.

28. The composition as in claim 26, wherein the non-fixed tissue sample is frozen when it is put into a hole in the housing block.
